(19) United States
(12) Patent Application Publication
(10) Pub. No.: US 2011/0237920 A1
(43) Pub. Date: Sep. 29, 2011

(54) SUCTION APPARATUS FOR EXTRACTING FLUID DURING A SURGICAL INTERVENTION

(76) Inventors: Simon Glaser, Eppelheim (DE); Philippe Federspil, Homburg(Saar) (DE); Peter K. Plinkert, Heidelberg (DE)

(21) Appl. No.: 13/058,751

(22) PCT Filed: Aug. 12, 2009
(86) PCT No.: PCT/EP09/05844

§ 371 Date: May 31, 2011

(30) Foreign Application Priority Data
Aug. 13, 2008 (DE) 10 2008 038 908.0

(51) Int. Cl. A61B 5/04; A61B 5/0488

(52) U.S. Cl. 600/373; 600/554

(57) ABSTRACT

A description is given of a suction apparatus for extracting fluid, in particular body fluid, during a surgical intervention, with a device for creating a suction pressure (60) and an extraction line (62), which is connected by its first end portion (61) to the device for creating the suction pressure and is provided on its second end portion (63) with a tubular suction connector (12), in order to pick up body fluid and carry it away through the extraction line. The suction apparatus has in this case a device for nerve stimulation with at least one electrode (13), by which a nerve tract can be electrically stimulated.

The present invention relates to a suction apparatus for extracting fluid, especially a bodily fluid, during a surgical procedure, comprising a device for generating a suction pressure and comprising an extraction line whose first end section is connected to the device for generating the suction pressure and whose second end section is provided with a tubular suction piece that serves to pick up fluid and to drain it through the extraction line. Moreover, the invention relates to a suction piece for such a suction apparatus.

Suction apparatuses are regularly used during surgical procedures in order to remove fluids, for example, blood flowing out of the wound during the operation. Such an apparatus can be used especially as a wound secretion suction apparatus. Normally, a suction piece is provided at the end of the extraction line facing the body, and its shape is adapted to the particular application purpose. Such suction pieces are known, among other things, by the names PLESTER suction cannula or BELUCCI suction tube, with or without a suction interrupter.

Moreover, it is a known technique to perform neurophysiological monitoring during a surgical procedure. As a result, neural structures of the body can be monitored during the operation and thus damage to the nerves due to the procedure can be avoided. In this manner, the functionality of motor nerves can be retained much better, especially when microsurgical techniques are employed, so as to avoid or reduce lesions caused by the surgery. Intraoperative neurophysiological monitoring is especially recommended for thyroid and parathyroid surgery, for neurosurgery, for head and throat surgery such as, for example, mastoidectomy, and for heart or chest surgery, but it can also be used for other operations.

Intraoperative neurophysiological monitoring functions according to the following principle: first of all, a nerve is directly stimulated using a probe to apply a small current surge of, for example, 0.2 mA to 2 mA. In the case of intact nerves, this triggers a muscle contraction that can be identified by means of leads on the basis of the resultant electric potentials, that can be electronically amplified and that can be indicated acoustically via a loudspeaker and/or visually on a display. The stimulation is carried out with either bipolar stimulation probes such as, for example, microfork probes and probes with ball tips, or else with unipolar probes. However, the prior-art stimulation probes are separate devices that are used independently of the suction apparatus.

The objective of the invention is to refine a suction apparatus and a suction piece for such an apparatus in such a way as to attain improved monitoring of the nerves during the surgical procedure.

This objective is achieved with a suction apparatus of the above-mentioned type by a device for nerve stimulation having at least one electrode by means of which a nerve path can be electrically stimulated.

The configuration of the suction apparatus according to the invention achieves an improved monitoring of the nerves. Since a device for nerve stimulation is provided on the suction piece, the suction piece can extract fluid and stimulate the nerves during surgery without a need to change instruments for this purpose. As a result, the monitoring can be carried out more quickly and more often than used to be the case. Furthermore, the invention allows an improved view of the operating field since, first of all, the number of instruments present in the operating field is reduced and secondly, fluid, for example, blood, can be picked up also during the stimulation. Moreover, the reduction of wound secretions or other fluids during the stimulation creates optimal conditions for nerve stimulation. This also contributes to a reduction of nerve injuries. The suction apparatus according to the invention can be used for all operations during which the nerves have to be monitored. In particular, the suction apparatus is suitable for thyroid, parotid, mastoid or neurosurgical operations. The suction piece can be configured, for instance, as a suction tube or as some other element having a suction opening that is operatively connected to the extraction line. The suction piece can be configured as a separate part or in one piece with the extraction line. Here, it is also possible for the suction piece to be formed by an end section of the extraction line. The extraction line can be configured to be partially or entirely flexible, as a result of which the suction piece can be easily moved into the desired position. The electric stimulation via the electrode can make use of direct currents, pulsed direct currents as well as alternating currents, which are preferably applied for only a short period of time. Here, low current intensities between 0.05 mA and 5 mA, preferably between 0.2 mA and 2 mA, are usually sufficient to achieve a measurable stimulation of an intact nerve.

According to an advantageous embodiment of the invention, it is provided that the at least one electrode is formed by at least one electrically conductive contact surface arranged on or in the suction piece. In this manner, an effective stimulation can be carried out, irrespective of whether fluid is being picked up by the suction piece during this time.

An especially good stimulation can be achieved without impairing the suction performance when the at least one contact surface is configured to be ring-shaped or partially ring-shaped. The same applies when the contact surface is arranged on the outer circumference of the suction piece.

Another improvement of the stimulation, along with a good suction performance, is achieved when the contact surface is configured to be circular or semicircular.

An especially precise and effective stimulation of the nerves is achieved if the at least one contact surface has one or more pins projecting from the suction piece. In particular, the pins can be arranged next to each other like the prongs of a fork. The ends of the pins can be configured to be pointed or rounded.

Another improvement is achieved in that the tubular suction piece has an electrically conductive end section, especially made of metal, that forms the electrode. In this context, it is possible to make only the electrically conductive end section or else the entire suction piece out of an electrically conductive material such as, for example, metal.

Moreover, it can be provided according to the invention that at least two contact surfaces are provided which especially are insulated from each other.

According to another particularly advantageous embodiment of the invention, it is provided that the at least one electrode is arranged on the suction piece so as to be adjustable, especially telescope-like. In this manner, the electrode can be moved into the desired position and an effective stimulation, along with a good suction performance, can be achieved even in varying spatial conditions.

In an advantageous manner, energy supply means can be provided by means of which a voltage can be applied to the at least one electrode. The energy supply means can be in the form of, for example, an energy storage means and/or at least one interface for an external energy supply means.

For this purpose, the energy supply means can comprise an electric line arranged on or in the suction piece, and said energy supply means is connected to the at least one electrode. This electric line can be configured as a single-strand or multi-strand line, depending on the intended application.

For purposes of attaining simple handling during the operation, the energy supply means can have a switch arranged especially on the suction piece in order to supply the at least one electrode with the voltage. In this manner, the surgeon can trigger the stimulation manually by pushing a button. As an alternative, of course, the stimulation can also be actuated by a foot switch or some other kind of switch.

Simple cleaning and sterilization are made possible if the suction piece containing the at least one electrode can be removed.

Moreover, it can be provided according to the invention that the electrode is configured as a unipolar or bipolar electrode. The bipolar configuration has the advantage that the calibration of the electrode is easier. In contrast, unipolar electrodes usually have to be calibrated manually.

The device for generating the suction pressure can advantageously be an electrically and/or mechanically driven device, for example, a pump. Of course, a vacuum line that is available in the operating room anyway can also be used as the device for generating suction pressure. In this manner, for example, blood, wound secretions, products of digestion, etc. can be extracted without any problem.

The risk of injuries during the suction procedure can be diminished if at least one bypass opening is formed in the suction piece. Such a bypass opening, which can be situated adjacent to the suction opening, allows non-traumatic suction.

The suction pressure can easily be controlled if the suction piece has at least one regulation opening. In this manner, the suction force can easily be adapted by opening and closing the regulation opening, for example, by laying a finger over it.

Moreover, the invention relates to a suction piece for the above-mentioned suction apparatus having a tubular section, whereby a device for nerve stimulation having at least one electrode for the electrical stimulation of a nerve path during a surgical procedure is arranged on the tubular section.

Additional objectives, features, advantages and application possibilities of the present invention ensue from the description below of embodiments making reference to the drawings. In this context, all of the described and/or depicted features on their own or in any desired combination are the subject matter of the invention, also their compilation in the individual claims and also irrespective of the claims to which they refer back.

The following is shown:

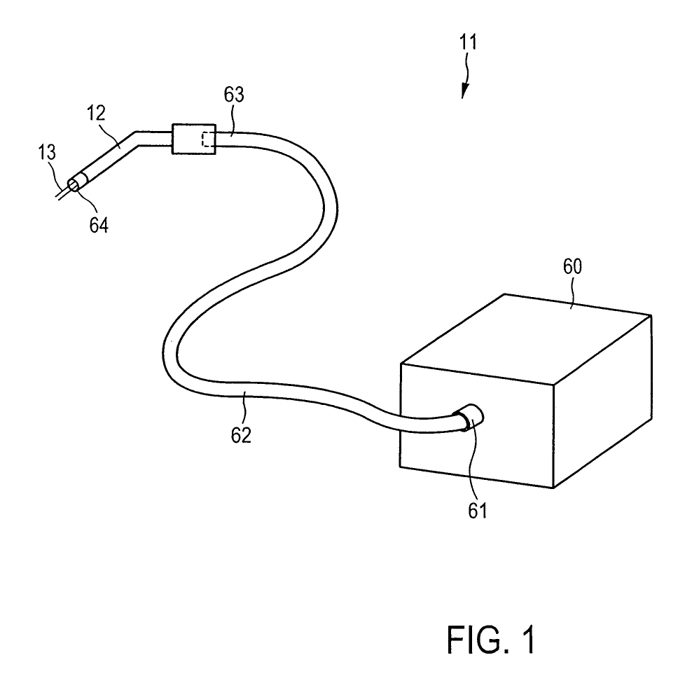

FIG. 1 a schematic depiction of a suction apparatus according to the invention,

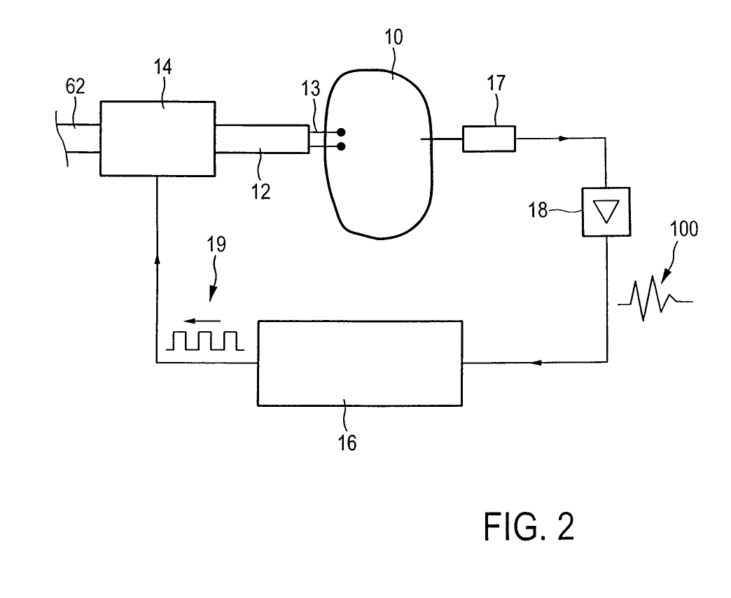

FIG. 2 a block diagram of a suction apparatus according to the invention, with a system for intraoperative neuro-monitoring;

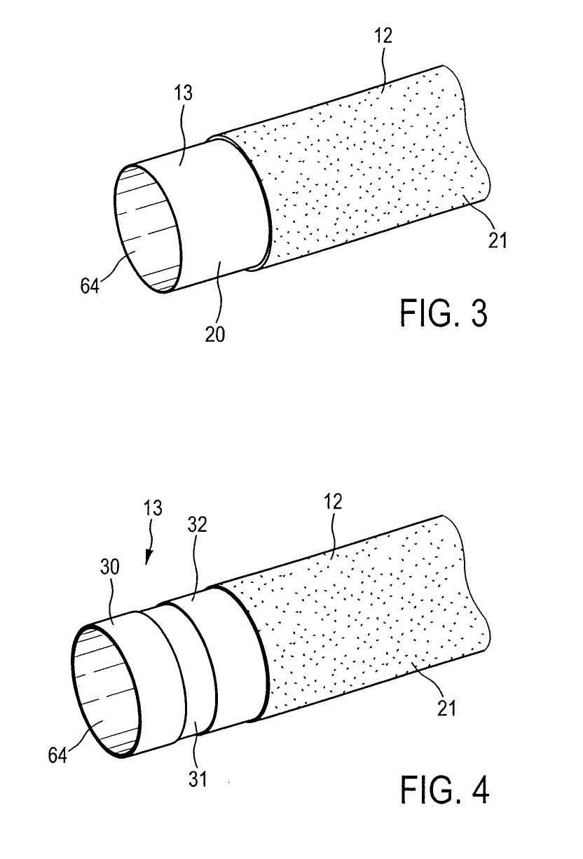

FIG. 3 a first variant of the suction piece of a suction apparatus according to the invention;

FIG. 4 a second variant of the suction piece of a suction apparatus according to the invention;

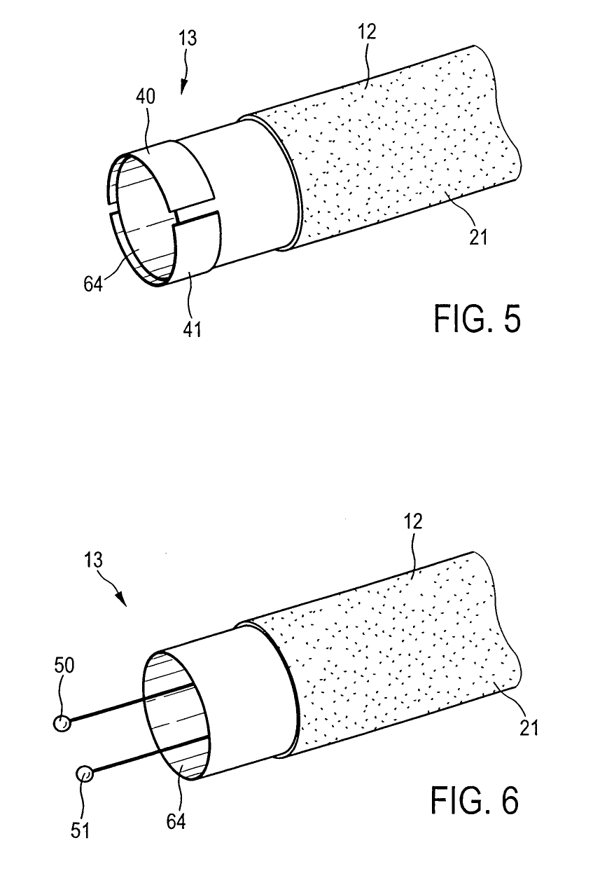

FIG. 5 a third variant of the suction piece of a suction apparatus according to the invention;

FIG. 6 a fourth variant of the suction piece of a suction apparatus according to the invention;

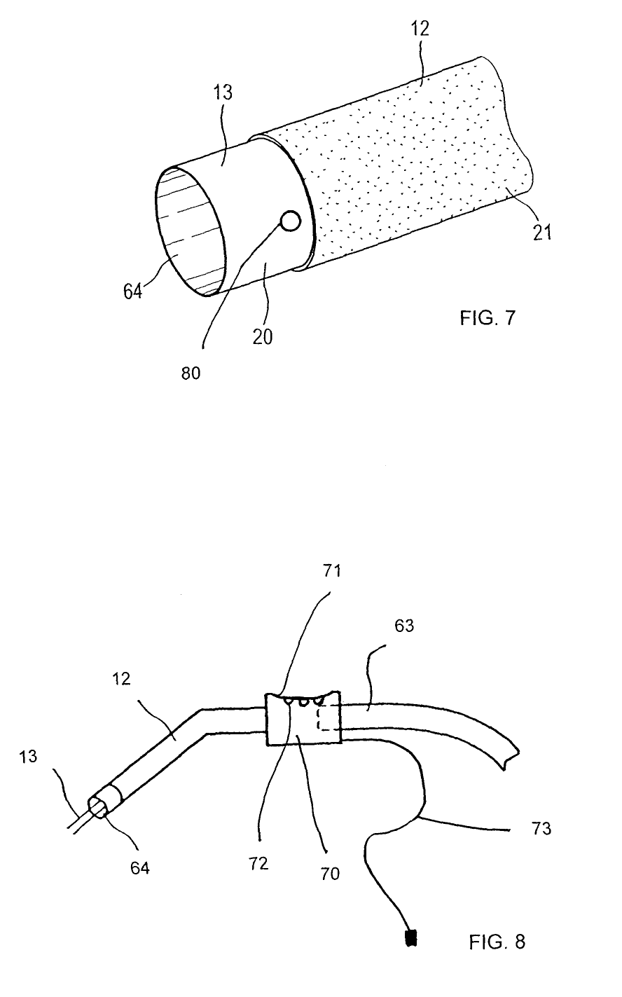

FIG. 7 a fifth variant of the suction piece of a suction apparatus according to the invention; and

FIG. 8 a partial depiction of another variant of a suction piece of a suction apparatus according to the invention, with regulation openings.

FIG. 1 shows a schematic depiction of a suction apparatus 11 according to the invention for extracting fluid, especially a bodily fluid, during a surgical procedure. The suction apparatus 11 has a device 60 for generating suction pressure which, in the embodiment shown, is configured as an electric vacuum pump. As an alternative, the suction pressure can also be provided in another manner. If a vacuum line is available in the operating room, it can be used to generate the suction pressure. An extraction line 62 is connected to the device 60 via a first end section 61. The extraction line 62 in the embodiment shown is movable, for example, as a flexible tube made of plastic, so that the second end section 63 can easily be moved into the operating field. On the second end section 63, there is a tubular suction piece 12 having a suction opening with which fluid can be extracted under the effect of the suction pressure. The suction piece 12 is detachably connected to the extraction line 62 and can be removed from it for purposes of cleaning and sterilization. However, the suction piece, in contrast to what is shown, can be configured in one piece with the extraction line 62 and/or can be formed by the second end section 63. The suction apparatus 11 also has electrodes 13 arranged on the suction piece 12, and these electrodes 13 are part of a device for nerve stimulation which is described in greater detail below. The tubular suction piece can be made partially or entirely of a rigid material. As an alternative, the tubular suction piece 12 can be made partially or entirely of a deformable material, for example, in the form of a tube.

FIG. 2 is a block diagram showing the configuration and arrangement of the device for nerve stimulation 16, which permits the intraoperative monitoring of nerves. Here, to start with, a body part 10, for example, a thyroid, is shown. Moreover, the schematically depicted suction piece 12 can be seen. During an operation, due to the vacuum generated by the device for generating a suction pressure, the suction piece 12 can pick up fluid, for example, blood and can transport it via an extraction line 62 to a collecting vessel (not shown here). For this purpose, a control unit 14 is provided for the extraction. The surgeon can trigger the extraction by means of an operating element (not shown here) that is provided on this control unit 14.

The device for nerve stimulation 16 has at least one electrode 13 arranged on the suction piece 12. The device for nerve stimulation 16 also has energy supply means to generate electric pulses 19 that are transmitted to the electrodes 13 via an electric line arranged in or on the extraction line 62. These electric pulses 19 stimulate nerves on the body part 10 as soon as the electrode is placed onto the nerve that is to be monitored. As a rule, a current surge between, for example, 0.2 mA and 2 mA is sufficient. In the case of an intact nerve path, the electric stimulation causes muscle contractions that can be detected by appropriate leads and converted into electric signals. For this purpose, a probe 17 which is configured as an insertion lead that can penetrate into the muscle to be monitored, is provided as the lead. A precise measurement is made possible since the probe 17 is inserted into the muscle. As an alternative, however, differently configured leads can be used for the electric recording. The signal generated in this manner is then pre-amplified by means of the amplifier 18 so that a signal 100 is available for further processing. As a function of the result of the analysis of this signal 100, an output is provided indicating whether the nerves are still reacting to the performed stimulation. This output can be provided acoustically and/or via display. This makes it possible for surgeons to already see the results of their procedures at an early point in time.

For the analysis, the device for nerve stimulation 16 has, for example, a processor to process the signal 100 and to generate a corresponding output signal for the acoustic and/or visual and/or haptic output. Moreover, a pulse generator is provided to generate the electric pulses 19. The device for nerve stimulation 16 can already comprise an acoustic output as well as a display, or if applicable, it can actuate such devices.

FIGS. 3 to 7 show five different variants of a suction piece 12 according to the invention. Each figure shows a partial view of the end of the tubular suction piece 12 facing the body. In the figures, the same reference numerals are used for parts that have the same function.

FIG. 3 shows a electrode 13 that is formed by a contact surface 20 arranged on the suction piece 12. The contact surface 20 is configured to be ring-shaped and it is arranged on the end of the tubular suction piece 12. In this manner, a unipolar electrode 13 is formed. The contact surface 20 is formed by an electrically conductive material, for example, metal, and the pulse 19 can be applied to it via an electric line (not shown here). The ring-shaped contact surface is arranged coaxially on the tubular section 21 of the suction piece and forms an end section of the suction piece 12 with the suction opening 64. The contact surface 20 is provided on the suction piece 12 in such a way that fluid can be picked up through the ring-shaped space delimited by the ring-shaped contact surface. The tubular section 21 can also be surrounded by an insulation layer 21 made, for example, of plastic. The insulation layer 21 can be configured, for instance, to be tube-like.

In the embodiment shown in FIG. 4, a bipolar electrode 13 is provided that has two contact surfaces 30, 32 arranged next to each other. Between the two coaxially arranged contact surfaces 30, 32, there is an insulating area that is configured here as an insulating ring 31. The contact surfaces 30, 32 both have a circular configuration and a matching diameter. The contact surfaces 30 and 32 are connected to separate lines that are electrically insulated from each other, and they can form a plus or minus pole of the electrode 13.

In the embodiment of the suction piece 12 shown in FIG. 5, two partially ring-shaped contact surfaces 40, 41 are provided for the electrodes 13. These contact surfaces 40, 41 are configured so as to be semicircular and separated from each other in the circumferential direction by an insulating material. As an alternative, there can be an insulating material between the two contact surfaces. The contact surfaces 40, 41 are electrically insulated from each other, they form a bipolar electrode 13 and are connected to lines (not shown here) in order to conduct the pulses 19 for the nerve stimulation all the way to the contact surfaces 40, 41.

In the embodiment shown in FIG. 6, the electrode 13 has pins 50, 51 that form the contact surfaces. In the embodiment shown, there are two pins 50, 51 that are configured like the prongs of a fork and arranged in parallel next to each other. The pins 50, 51 project from the suction piece 12 and are attached inside the tubular suction piece 12. They extend from the suction opening 64 and project over the tubular edge of the suction piece 12. The electrode 13 configured here as pins 50, 51 can be arranged adjustably on the suction piece so that it assumes a more or less protruding position relative to the tubular section 21. An operating element (not shown here), which can be configured, for example, as a slide, can be provided to easily adjust the electrode 13. In the embodiment shown, the pins are rounded at their outer end and, for this purpose, they have an expanded, especially spherical section.

The embodiment shown in FIG. 7 shows a suction piece 12 with a bypass opening 80. The bypass opening 80 is configured in the wall of the suction piece 12 adjacent to the suction opening 64. The bypass opening 80 allows non-traumatic suction, even under difficult conditions. When the suction opening 64 comes to lie on the surface of an organ and is consequently closed, air or fluid can still be drawn in through the bypass opening 80. The bypass opening 80 can preferably be arranged 2 mm to 4 mm from the edge of the suction opening 64. Advantageously, several bypass openings 80 can be arranged on the circumference of the suction piece 12, for example, there can be four bypass openings that are distributed over the circumference. The entire cross sectional surface area of the bypass opening 80 or bypass openings 80 is smaller than the cross sectional surface area of the suction opening 64. For the rest, the embodiment shown in FIG. 7 corresponds to the embodiment shown in FIG. 3. The description of the latter applies correspondingly to FIG. 7.

FIG. 8 shows an embodiment of the suction apparatus with an angled suction piece 12 having a grip section 70. The grip section 70 is provided with a gripping surface 71 that, in the embodiment shown, is curved towards the inside. The gripping surface 71 has regulation openings 72 that connect the inside of the suction piece 12 to the environment. When the regulation openings 72 are closed or opened during their use, for example, with a finger, the suction action can be regulated simply and quickly. FIG. 8 also shows an electric connection line 73 that is connected to the electrode 13.

In all of the embodiments, the suction piece 12 can be configured as a snap-on part that can be detachably connected to the suction line 62. The suction piece 12 with the contact surfaces can then be removed for cleaning purposes and sterilized, for example, in an autoclave. Moreover, the suction piece can have a suction bypass opening (not shown here) that allows the surgeon to manually regulate the suction force that is present at the suction opening 64.

### LIST OF REFERENCE NUMERALS

10 body part
11 suction apparatus for extracting bodily fluid
12 suction piece
13 electrode
14 control unit for the extraction
16 device for nerve stimulation
17 probe
18 pre-amplifier
19 pulse
100 signal
20 contact surface
21 tubular section
30 contact surface
31 insulating ring
32 contact surface
40 contact surface
41 contact surface
50 contact surface
51 contact surface
60 device for generating a suction pressure
61 first end section
62 extraction line
63 second end section
64 suction opening
70 grip section
71 gripping surface
72 regulations openings
73 connection line
80 bypass opening

1. A suction apparatus for extracting fluid, especially a bodily fluid, during a surgical procedure, comprising
a device for generating a suction pressure and comprising an extraction line whose first end section is connected to the device for generating the suction pressure and whose second end section is provided with a tubular suction piece that serves to pick up fluid and to drain it through the extraction line, and
a device for nerve stimulation having at least one electrode for electrically stimulating a nerve path.

2. The suction apparatus according to claim 1, wherein the at least one electrode is formed by at least one electrically conductive contact surface arranged on or in the suction piece.

3. The suction apparatus according to claim 2, wherein the at least one contact surface is configured to be ring-shaped or partially ring-shaped.

4. The suction apparatus according to claim 2, characterized wherein the contact surface is arranged on the outer circumference of the suction piece.

5. The suction apparatus according to claim 2, wherein the contact surface is configured to be circular or semicircular.

6. The suction apparatus according to claim 2, wherein the at least one contact surface has one or more pins projecting from the suction piece.

7. The suction apparatus according to claim 2, wherein the tubular suction piece has an electrically conductive end section, especially made of metal, that forms the electrode.

8. The suction apparatus according to claim 2, wherein at least two contact surfaces are provided which especially are insulated from each other.

9. The suction apparatus according to claim 2, wherein the at least one electrode is arranged on the suction piece so as to be adjustable telescopically.

10. The suction apparatus according to claim 2, wherein energy supply means are provided by means of which a voltage can be applied to the at least one electrode.

11. The suction apparatus according to claim 10, wherein the energy supply means comprises an electric line arranged on or in the suction piece, and said energy supply means is connected to the at least one electrode.

12. The suction apparatus according to claim 10, wherein the energy supply means has a switch arranged especially on the suction piece in order to supply the at least one electrode with voltage.

13. The suction apparatus according to claim 1, wherein the suction piece containing the at least one electrode can be removed.

14. The suction apparatus according to claim 1, wherein the at least one electrode is configured as a unipolar or bipolar electrode.

15. The suction apparatus according to claim 1, wherein at least one bypass opening is formed in the suction piece.

16. The suction apparatus according to claim 1, wherein the suction piece has at least one regulation opening.

17. A suction piece for the suction apparatus according to claim 1, having a tubular section, characterized by a device for nerve stimulation that has at least one electrode for the electrical stimulation of a nerve path during a surgical procedure and that is arranged on the tubular section.

18. The suction apparatus according to claim 1, wherein the at least one electrode comprises a lead for detecting the resultant muscle contractions.
